(19) United States
(12) Patent Application Publication
(10) Pub. No.: US 2003/0220661 A1
(43) Pub. Date: Nov. 27, 2003

(54) Transmyocardial implant delivery system

(75) Inventors: David H. Mowry, Waconia, MN (US); Robert Edward Kohler, Lake Elmo, MN (US)

Correspondence Address: MERCHANT & GOULD PC, P.O. BOX 2903, MINNEAPOLIS, MN, 55402-0903, US

(73) Assignee: HeartStent Corporation

(21) Appl. No.: 10/153,341

(22) Filed: May 21, 2002

(51) Int. Cl. A61B 17/08

(52) U.S. Cl. 606/153; 606/108; 606/205; 623/1.36; 623/1.1

(57) ABSTRACT

A transmyocardial implant delivery system is disclosed for establishing a blood flow path through a myocardium between a heart chamber and a lumen of a coronary vessel residing on an exterior of the heart. The transmyocardial implant delivery assembly includes as conduit for defining a blood flow path, a temporary sheath, and a tool holding a second end of the conduit. The conduit includes a rigid portion adapted to be inserted into and retained within the heart wall of a heart chamber. The rigid conduit is sufficiently rigid to withstand collapsing in response to contraction forces of the heart wall. The conduit also includes a synthetic flexible portion that is in fluid communication with the rigid portion. An end of the flexible portion corresponding to the second end of the conduit is sized to be received in a lumen of a coronary vessel. The transmyocardial implant delivery system is inserted through the heart wall. After insertion the second end of the conduit is communicated with a lumen of a coronary vessel. The sheath can then be removed leaving the conduit in place within the heart wall.

### BACKGROUND OF THE INVENTION

1. Field of the Invention

The present invention relates to cardiac revascularization devices, and more particularly, to a transmyocardial implant delivery system and corresponding method for forming a blood flow path through a heart wall from a heart chamber to a coronary vessel.

2. Description of the Prior Art

U.S. Pat. No. 5,944,019, issued Aug. 31, 1999, the disclosure of which is incorporated herein by reference, teaches an implant for defining a blood flow conduit directly from a chamber of the heart to a lumen of a coronary vessel. One embodiment disclosed in the aforementioned patent teaches an L-shaped implant in the form of a rigid conduit having one leg sized to be received within a lumen of a coronary artery and a second leg sized to pass through the myocardium and extend into the left ventricle of the heart. As disclosed in the above-referenced patent, the conduit is rigid and remains open for blood flow to pass through the conduit during both systole and diastole. The conduit penetrates into the left ventricle in order to prevent tissue growth and occlusions over an opening of the conduit. Other embodiments are also disclosed wherein a rigid portion in within the heart wall and a flexible portion is within the lumen of the coronary artery.

### SUMMARY OF THE INVENTION

The present invention relates to a transmyocardial implant delivery system for establishing a blood flow path through a myocardium between a heart chamber and a coronary vessel residing on an exterior of the heart.

A variety of advantages of the invention will be set forth in the description which follows, and will be apparent from the description. It is to be understood that both the foregoing material and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate several aspects of the invention and together with the description, serve to explain the principles of the invention. A brief description of the drawings is as follows:

FIG. 1 is front view of a transmyocardial implant delivery system that is an embodiment of the present invention;

FIG. 2 is a front view of the transmyocardial implant delivery system depicted in FIG. 1 showing an inner conduit partially deployed from an outer sheath;

FIG. 2A is a right side view of the implant delivery system of FIG. 1;

FIG. 3 is a cross-sectional view of the transmyocardial implant depicted in FIG. 1;

FIG. 4 is a longitudinal cross-sectional view of an anastomosis device in an expanded orientation incorporated into an implant according to the present invention;

FIG. 5 is a longitudinal cross-sectional view of the anastomosis device of FIG. 4 in a partially compressed orientation;

FIG. 6 is an end view of the anastomosis device of FIG. 4;

FIG. 7 is a cross-sectional view of an alternative anastomosis device shown in a compressed orientation;

FIG. 8 is a cross-sectional view of the alternative anastomosis device shown in FIG. 7 in an expanded orientation;

FIG. 9 is a side sectional view of the alternative anastomosis device of FIG. 7 showing anchoring teeth of the device embedded in a vessel wall;

FIG. 10 is a plan view of a tool that can be used as part of a system or kit in accordance with the principles of the present invention, a working end of the tool is shown open;

FIG. 11 is a plan view of the tool of FIG. 10 with the working end closed;

FIG. 12 is a schematic view of a transmyocardial implant delivery assembly surgical kit that is an embodiment of the present invention;

FIG. 13 is a plan view of an obstructed coronary artery lying on an outer surface of a heart wall;

FIG. 14 is a side sectional view of the coronary artery of FIG. 13 showing the artery, obstruction and a myocardium in cross-section;

FIG. 15 is a plan view of the obstructed coronary artery of FIG. 13, with the transmyocardial implant delivery system of FIG. 1 inserted through the myocardium.

FIG. 16 is a side sectional view of the coronary artery of FIG. 15.

FIG. 17 is a side sectional view of the coronary artery of FIG. 16, with the sheath of the transmyocardial implant delivery system withdrawn from the myocardium.

FIG. 18 is a cross-sectional view of the coronary artery of FIG. 16, with the transmyocardial implant of FIG. 2 connecting the heart chamber and the coronary vessel.

### DETAILED DESCRIPTION

Reference will now be made in detail to exemplary aspects of the present invention that are illustrated in the accompanying drawings. The same reference numbers will be used throughout the drawings to refer to the same or like parts. It should be noted that throughout the description, the terms “including”, “having”, and “containing” are used synonymously with “comprising”. The term “myocardium” is used interchangeably with “heart wall”. Additionally, the terms “implant”, “conduit”, and “shunt” will also be used interchangeably throughout the description. It will be understood that the implant delivery assembly described herein can be used to delivery a conduit into the heart wall at any position so as to form a blood flow passage for the flow of blood from any heart chamber.

The present invention is described with reference to placement of a transmyocardial implant 110 between a coronary artery and a left ventricle. It will be appreciated the invention is applicable to the formation of a direct blood flow path between a heart chamber (left or right ventricle or atrium) and a coronary vessel (artery, vein, or a branch thereof). Further, as vessel size and myocardium thickness vary throughout the heart, the size of implant 110 will vary depending upon a vessel selected for a procedure and a myocardium thickness.

I. Transmyocardial Implant Delivery Assembly and Kit

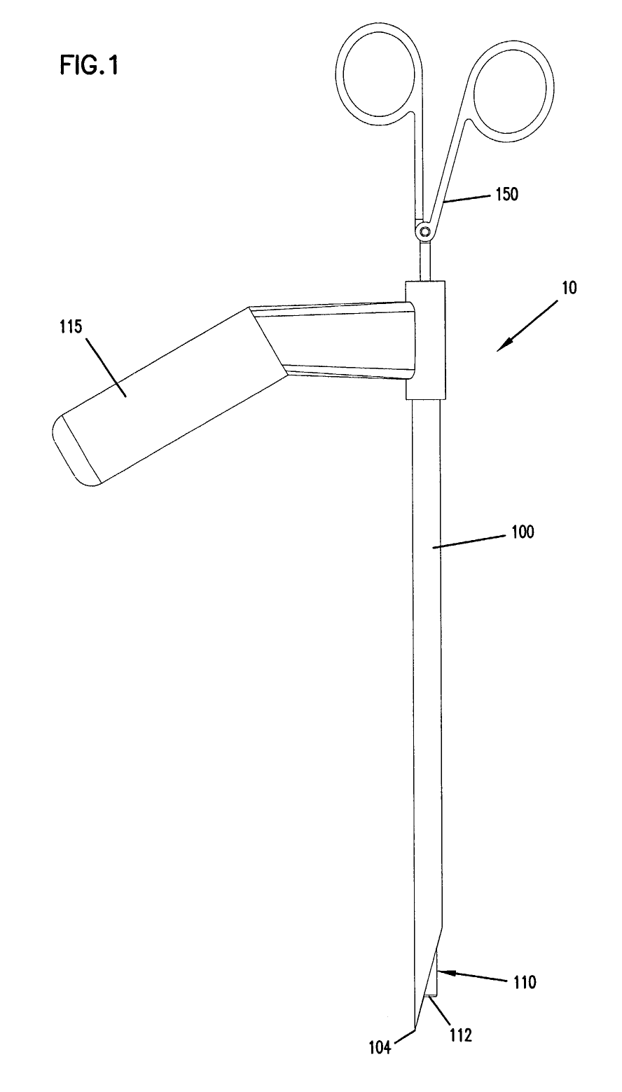

FIGS. 1 through 3 show a transmyocardial implant assembly 10 that is an example embodiment of the present invention. Implant assembly 10 includes a conduit or implant 110 surrounded by sheath 100. Implant assembly 10 also includes a tool 150 connected to a second end 114 of conduit 110. In one embodiment, tool 150 is adapted for grasping second end 114 of conduit 110. Additionally, implant assembly 10 may include a handle 115 that projects outwardly from sheath 100.

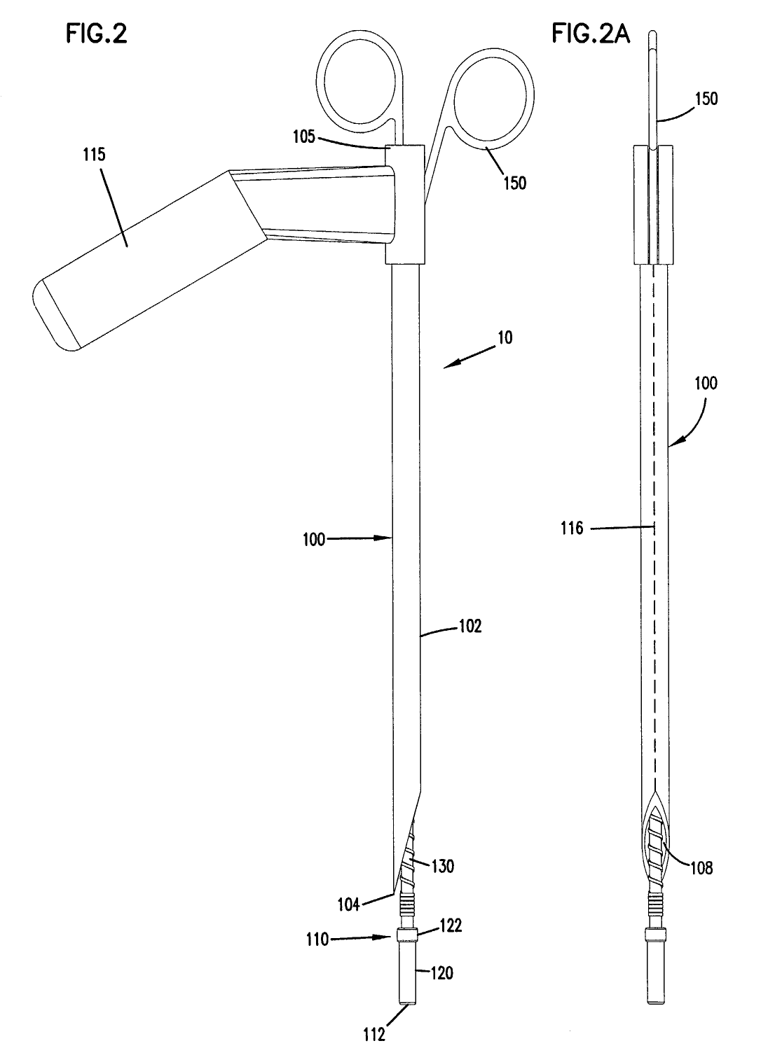

Referring to FIG. 2, conduit 110 includes a rigid portion 120 and a flexible portion 130. It will be appreciated that the term “rigid” means that rigid portion 120 is sufficiently rigid so as to withstand the contraction forces of the myocardium and remains open during both systole and diastole. Rigid portion 120 may be formed of any suitable material. In one embodiment, rigid portion 120 is formed of low density polyethylene (LDPE). Alternatively, rigid portion 120 may be formed of titanium, a nickel-titanium alloy or another rigid biocompatible material such as pyrolytic carbon including titanium coated with pyrolytic carbon or another anti-thrombotic material such as parylene. A first end 112 of rigid portion 120 (also referred to as first end 112 of the conduit 110) is sized to extend through the myocardium of the human heart to project into the interior of a heart chamber (preferably, the left ventricle) by a suitable distance such that the blood flow path will remain open when the heart wall thickens during contraction and to prevent the heart wall from growing over first end 112.

Flexible portion 130 is in fluid communication with rigid portion 120. Flexible portion 130 is preferably made from any suitable plastic material, preferably, expanded polytetrafluoroethylene (ePTFE). The use of ePTFE is advantageous since this is a material already used as a synthetic vessel with proven blood and tissue compatibility thereby reducing risk of thrombosis and encouraging endotheliazation. Second end 114 of the flexible portion 130 (also referred to as second end 114 of the conduit 110) is sized to be received within the lumen of a coronary vessel, such as a coronary artery.

To form an implant 110, an end of flexible portion 130 opposite second end 114 may be inserted into the interior of the rigid portion 130 through an end opposite first end 112 and secured. Flexible portion 130 may be secured in rigid portion 130 using collagen or another suitable polymer, or may be secured using a forced fit. Alternatively, when rigid portion 120 is formed of LDPE, flexible portion 130 may be secured to rigid portion 120 by heat bonding along all surfaces of opposing material of rigid portion 120 and flexible portion 130. At elevated temperatures, the material of rigid portion 120 flows into the micro-pores of the material of flexible conduit 130. In this embodiment, rigid portion 120 will have a lower melting point than the flexible material.

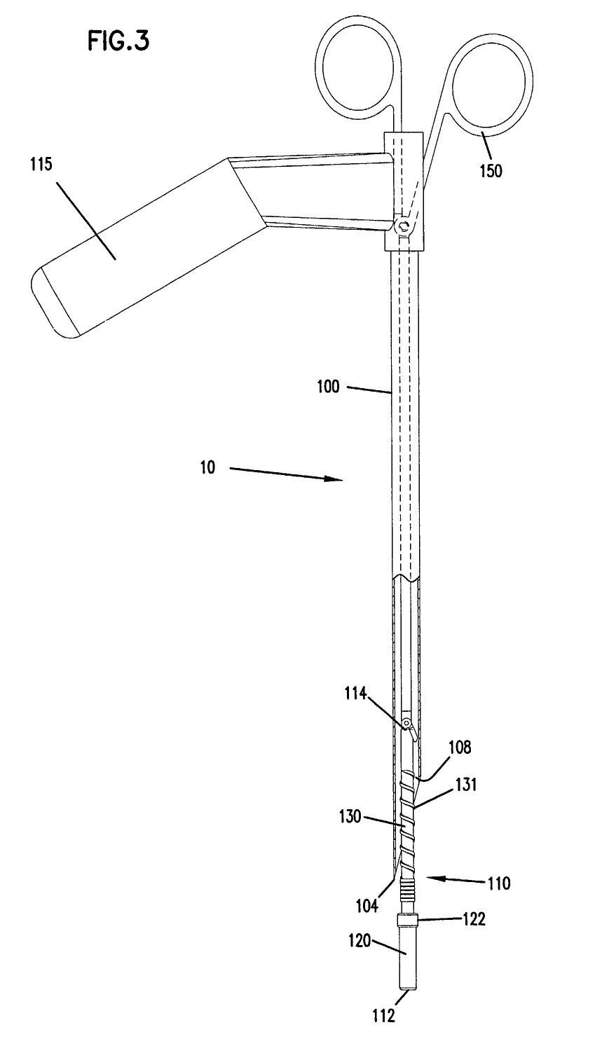

A plurality of support rings 131, as shown in FIG. 3, may be provided along the length of flexible portion 130 of conduit 110 not otherwise attached to or supported by rigid portion 120. Preferably, rings 131 are LDPE each having an interior surface heat bonded to an exterior surface of flexible portion 130. At their inner radius, LDPE rings 131 can be integrally formed with the outer radius of flexible portion 130 with the cross-sectional planes of rings 131 set at a fixed angle of separation (e.g., about 20 degrees) to support flexible portion 130 throughout a 90 degree bend. Rings 131 provide crush resistance to flexible portion 130. Between the rings flexible portion 130 may flex inwardly and outwardly to better simulate the natural compliance of a natural blood vessel. As shown at FIG. 3, rings 131 include relatively widely spaced rings 131a and relatively closely spaced rings 131b. By way of a further non-limiting example, helical rings 131 could be replaced with a discrete ring. In other embodiments, the rings could be eliminated.

Transmyocardial implant delivery assembly 10 including conduit 110 is preferably inserted into the myocardium through sheath 100 such that end 112 of rigid portion 120 of conduit 110 protrudes into the heart chamber. Conduit 110 defines an open blood flow path that allows blood flow communication directly between the left ventricle and a lumen of a coronary vessel lying at an exterior of the heart wall. To bypass an obstruction in a coronary artery, end 114 of flexible portion 130 of implant 110 is attached to the artery. For example, end 114 may be anastomosed to the artery in an end-to-side anastomosis with an anastomosis device 140 or may be secured by suturing. End 114 is secured to the artery distal (i.e., downstream from) to the obstruction. End-to-end connection techniques are also within the scope of the present invention.

A sleeve 122 may surround rigid portion 120. Preferably, such a sleeve would be formed of a fabric having biocompatible fibers defining interstitial spaces to receive tissue growth. An example of such a fabric is polyethylene terephthalate (such as polyester fabric sold by DuPont Company under the trademark DACRON). Such a fabric permits rapid tissue integration into the fabric to anchor the fabric and, hence, implant 110 to the patient's tissue.

End 114 of flexible portion 130 of implant 110 may also comprise an anastomosis device 140 for end to side anastomosis with a coronary vessel. Shown separately in FIGS. 4-9, anastomosis device 140 includes a flange 142 positioned at second end 114 of flexible portion 130 of conduit 110. Flange 142 includes a main body 143 that is integrally formed (i.e., unitarily or monolithically formed as a common, seamless piece) with end 114 flexible portion 130 of conduit 110. For example, main body 143 of flange 142 and flexible portion 130 of conduit 110 can be integrally formed of ePTFE. Alternatively, flange 142 can be a separate piece that is bonded or otherwise secured to second end 114 of conduit 110.

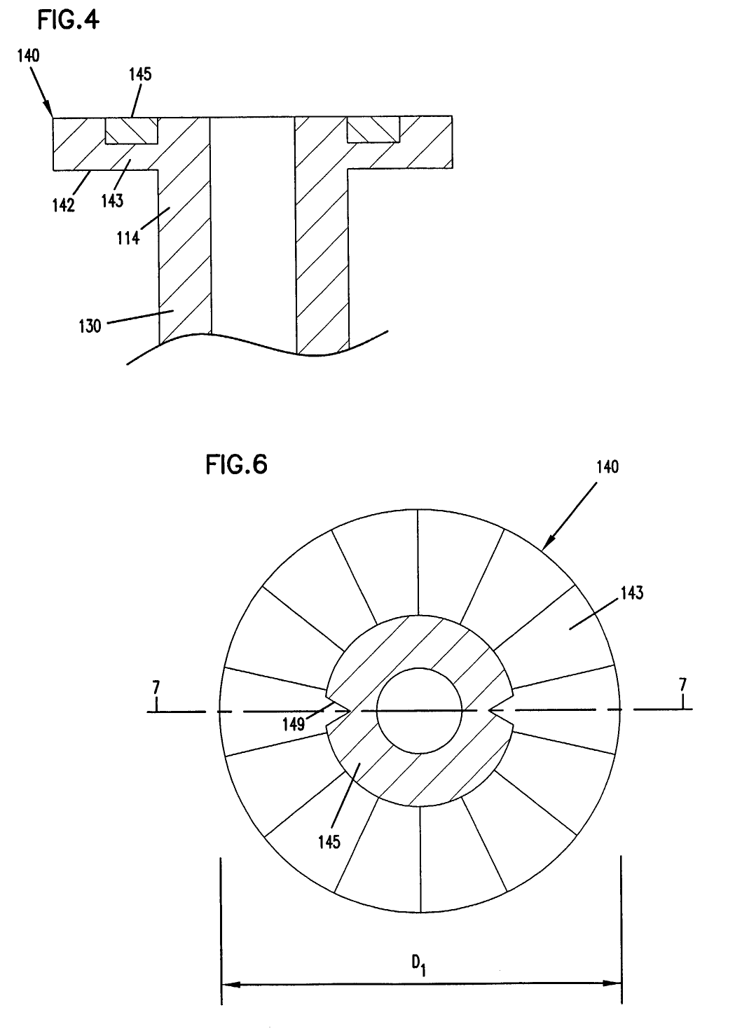

Flange 142 is movable between an expanded orientation (shown in FIG. 4) and a compressed orientation (shown partially compressed in FIG. 5). In the expanded orientation, flange 142 projects radially outwardly from flexible portion 130 of conduit 110 and has an enlarged shape or perimeter. For example, flange 142 circumferentially surrounds (i.e., concentrically surrounds) end 114 of conduit 110 and has a generally circular shape (shown in FIG. 6). Preferably, flange 142 has an outer diameter larger than the outer diameter of flexible conduit 130. In one embodiment, flange 142 has an outer diameter in the range of 10% to 100% larger than the outer diameter of flexible portion 130. While a circular shape is preferred, other shapes such as elliptical shapes and oblong shapes could also be used. Jointly owned U.S. patent application Ser. No. 09/686689, filed on Oct. 11, 2000, the disclosure of which is incorporated herein by reference, shows auto-anastomosis devices that might be incorporated in the present invention as flange 142. Further, for certain applications it may be desirable to use a non-rounded shape (e.g., square).

Flange 142 preferably includes a biasing structure for resiliently biasing (i.e., in a spring-like manner) flange 142 toward the expanded orientation. For example, the resilient structure can be provided by the inherent properties of the materials selected to make main body 143 of flange 142. Alternatively, a separate resilient structure can be connected to (i.e., embedded in, bonded to, fastened to, or otherwise secured to) the main body of flange 142. For example, FIG. 5 shows a resilient structure in the form of a resilient ring 145 embedded in main body 143 of flange 142. Ring 145 is preferably made of an elastic or super elastic material. In one embodiment, ring 145 is made of a metal that exhibits elastic or super elastic characteristics such as a nickel titanium alloy.

As shown in FIG. 5, flange 142 is moved to the compressed orientation by folding flange 142 upwardly about a fold line 147. In alternative embodiments, flange 142 could also be folded downwardly about fold line 147. Fold line 147 can be defined by a hinge 149 (e.g., regions of reduced thickness) provided on ring 145 (see FIG. 6). When moved to the compressed orientation, flange 142 is folded about fold line 147 into two generally semi-circular halves. With flange 142 oriented in the compressed configuration, an outer diameter D₁ (labeled in FIG. 6) in a direction taken along fold line 147 is equal to the outer diameter of expanded flange 142. However, when in the compressed orientation, an outer diameter D₂ (labeled in FIG. 5) in a direction that is transverse relative to fold line 147 is substantially reduced as compared to the outer diameter of expanded flange 142. By reducing the diameter in at least one direction, flange 142 can be passed through a vessel incision having a size approximately the same as the outer diameter of flexible portion 130. This can be accomplished by manipulating flexible portion 130 relative to the vessel such that a first end of fold line 147 is initially inserted through the opening, and the opposite end of fold line 147 is subsequently passed through the incision. During the insertion process, flange 142 is preferably held in the compressed orientation by a retaining tool such as a forceps or a retractable sheath or collar. If a tool 150 such as a forceps is used as is depicted with the implant assembly 10 in FIGS. 1-3, the physician uses forceps 150 to manually hold flange 142 in a folded orientation until after insertion in the vessel. Once flange 142 has been inserted within the vessel, flange 142 can be released from retaining tool 150 thereby allowing flange 142 to self-expand to the expanded orientation within the vessel. Flange 142′ may further include barbs 166 to secure device 140′ to the vessel.

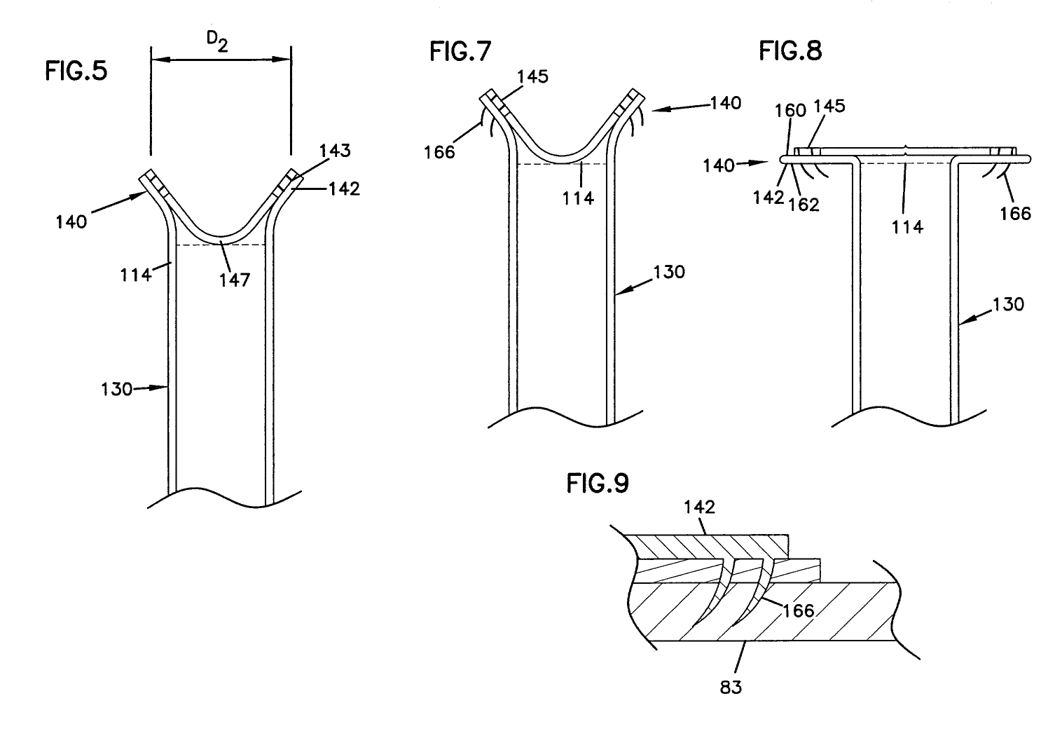

Transmyocardial implant assembly 10 as depicted in FIGS. 1-3 also includes sheath 100. Sheath 100 is temporary and is preferably capable of being torn away from conduit 110 once assembly 10 has been inserted into the myocardium. For example, as shown in FIG. 2A, sheath 100 can include a part-line 111 (e.g., a scored or perforated line). An example sheath having a tear-away feature is disclosed in U.S. Pat. No. 6,029,672, issued Feb. 29, 2000, the disclosure of which is incorporated herein by reference. In one embodiment, sheath 100 may be configured so as to surround implant 110 including rigid portion 120 and flexible portion 130, as illustrated in FIGS. 1-3. As shown by the alternative anastomosis device 140′ of FIGS. 7-9, sheath 100 may be configured so as to partially surround implant 110, for example, enclosing only rigid portion 120 of implant 110.

In one non-limiting embodiment, sheath 100 has a plastic (e.g., polytetrafluoroethylene) hollow cylindrical body 102 terminating at a distal tip 104. Handle 115 is attached adjacent a proximal end 105 of heath 100 to permit grasping by a surgeon. Tip 104 may be blunt or may be adapted to penetrate and to form a bore through the myocardium. The apex of tip 104 can be closed (not shown) or, more preferably, provided with a through-hole 108. In another embodiment (not shown), through-hole is slightly larger in diameter than a guide wire or catheter to be delivered into the heart. As such, a guide wire or catheter may be in place during insertion of transmyocardial implant delivery assembly 10 at a desired location in the myocardium to guide insertion of sheath 100 into the heart wall.

The inside diameter of body 102 is sized to receive implant 110. In one embodiment, implant 110 is pressed within sheath 100. In this embodiment, after placement of rigid portion 120 of implant 110 within myocardial sheath 100, rigid portion 120 or sleeve 122 acts as a gasket to seal against the interior surface of body 102 to prevent blood flow between sheath 100 and implant 110.

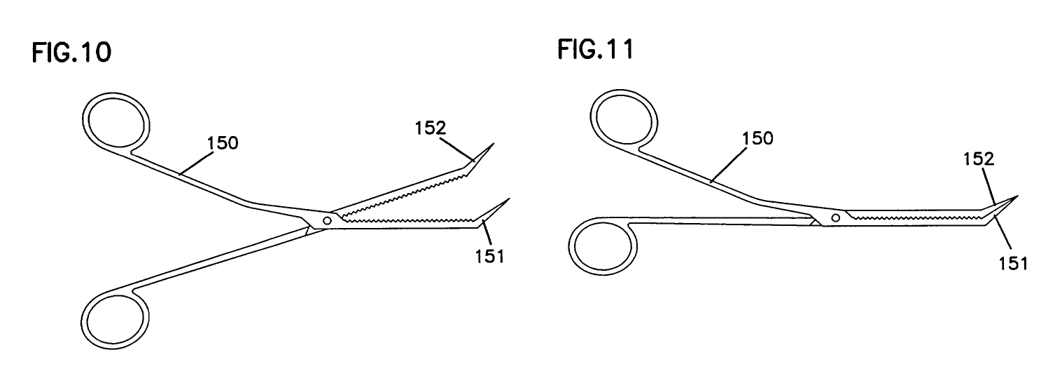

Implant assembly 10 also includes a tool 150 grasping second end 114 of conduit 110 (FIGS. 1-3). Tool 150 may be any suitable tool with a working end 151 for grasping or otherwise holding or connecting to end 114 of the conduit. Handle 115 may also be provided. Handle 115 holds tool 150, conduit 110, and sheath 100 in placement for rapid delivery into the myocardium. Preferably, tool 150 is a forceps 150 as shown in FIGS. 10 and 11. A surgeon may manipulate tool 150 to facilitate insertion of implant 110. If handle 115 is present, the surgeon may grasp handle 115 with one hand and tool 150 with the other, and may manipulate tool 150 to facilitate insertion of implant delivery system 10 through the myocardium into the heart chamber. Tool 150 may include a blade 152 at working end 151 (best seen in FIG. 11). Blade 152 located at working end 151 of tool 150 facilitates anastomosis of second end 114 of implant 110 with a lumen of a coronary vessel by allowing the incision and anastomosis to be completed with the same tool in a rapid, almost simultaneous, succession of steps.

As previously indicated with respect to FIG. 2A, body 102 and tip 104 may have an axially extending part-line 111 on a side of body 102 opposite handle 115. Part-line 111 may be a score partially or totally through the wall thickness or perforations through the wall thickness. Part-line 111 permits sheath 100 to be split open along its axial length. Body 102 and tip 104 are flexible to spread apart at part-line 111 by a separation sufficient to pass sheath 100 over implant 100 as will be described. Alternatively, sheath 100 simply may be withdrawn intact from the myocardium.

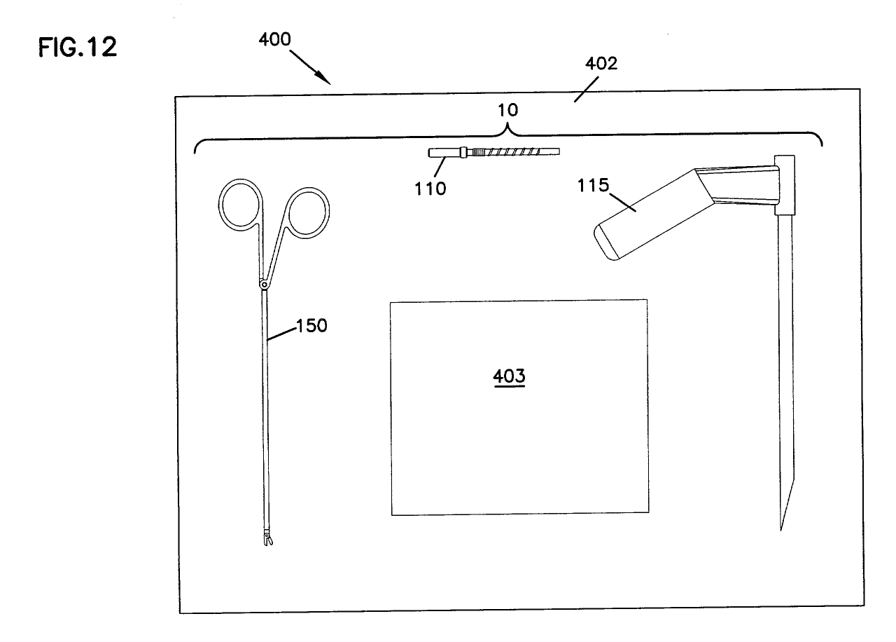

Referring to FIG. 12, implant delivery assembly 10 may be provided as part of a sterile, sealed package or kit. An exemplary kit 400 may include a suitable container 402, (e.g., a sealed bag). Implant delivery system 10 and instructions 403 for use are preferably within the container 402. Often kits can be provided with multiple implants (e.g., implants of different lengths, diameters or connection configurations). In one embodiment, implants can be provided with and without autoanastomosis devices. The implants without autoanastomosis devices can be cut to length at the time of surgery. Other kits can include multiple sheaths or multiple types of insertion tools.

II. Procedure

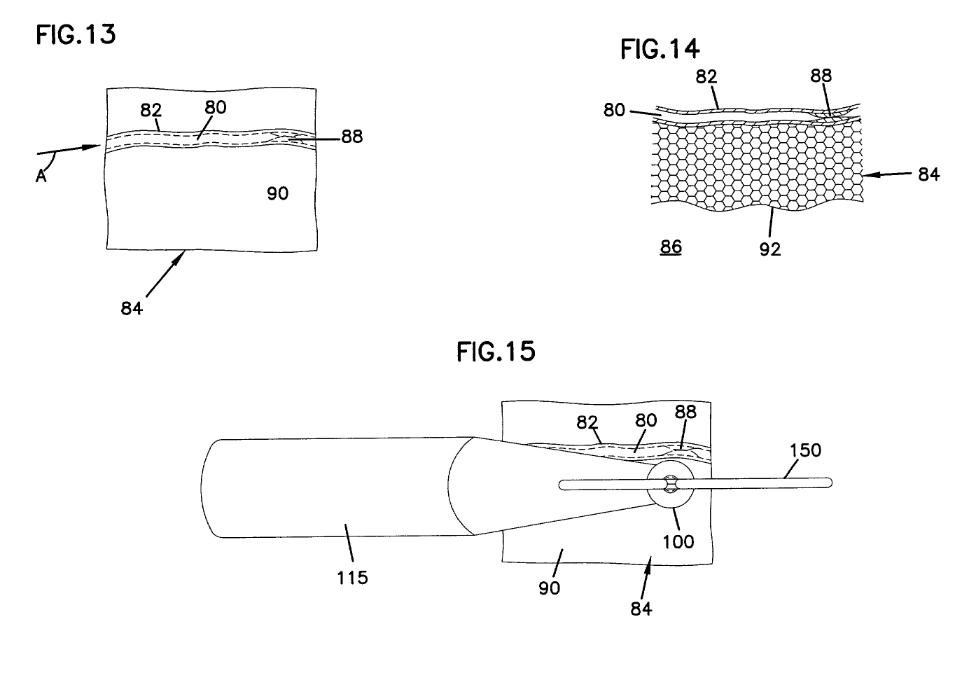

The procedure of the present invention is illustrated in FIGS. 13 through 18. FIG. 13 is a plan view of an exterior surface 90 of a heart wall 84 with a coronary vessel 82 lying on the surface 90. A lumen 80 of vessel 82 is shown in phantom lines. The present procedure is applicable for use in a wide number of coronary vessels. For ease of discussion, the invention will be described with reference to vessel 82 being a coronary artery (e.g., LAD) on the left side of the heart overlying a left ventricle 86. Normal blood flow through artery 82 is in the direction of arrow A. Such blood flow is at least partially obstructed by an occlusion 88. FIG. 14 is a cross-sectional view of FIG. 13 showing an interior surface 92 of heart myocardium 84 and left ventricle 86.

The procedure can be initiated by first determining a thickness of the heart wall at the desired implantation site. This can be done by inserting an externally graduated needle through the heart wall to measure the thickness. Example measuring techniques are disclosed in U.S. Pat. No. 6,193,726, issued Feb. 28, 2001, the disclosure of which is incorporated herein by reference.

As implant 110 may be provided as part of a delivery assembly 10, no separate step may be required for placing implant 110 in sheath 100. Delivery assembly 10 may be available to the surgeon as a single pre-assembled unit or after determining the thickness of myocardial 84 the surgeon may customize delivery assembly 10 by selecting the various components depending upon the patient and the nature of the procedure. While kit 400 described above is designed for this purpose, it is not required in order for the surgeon or other person to assemble implant delivery system 10.

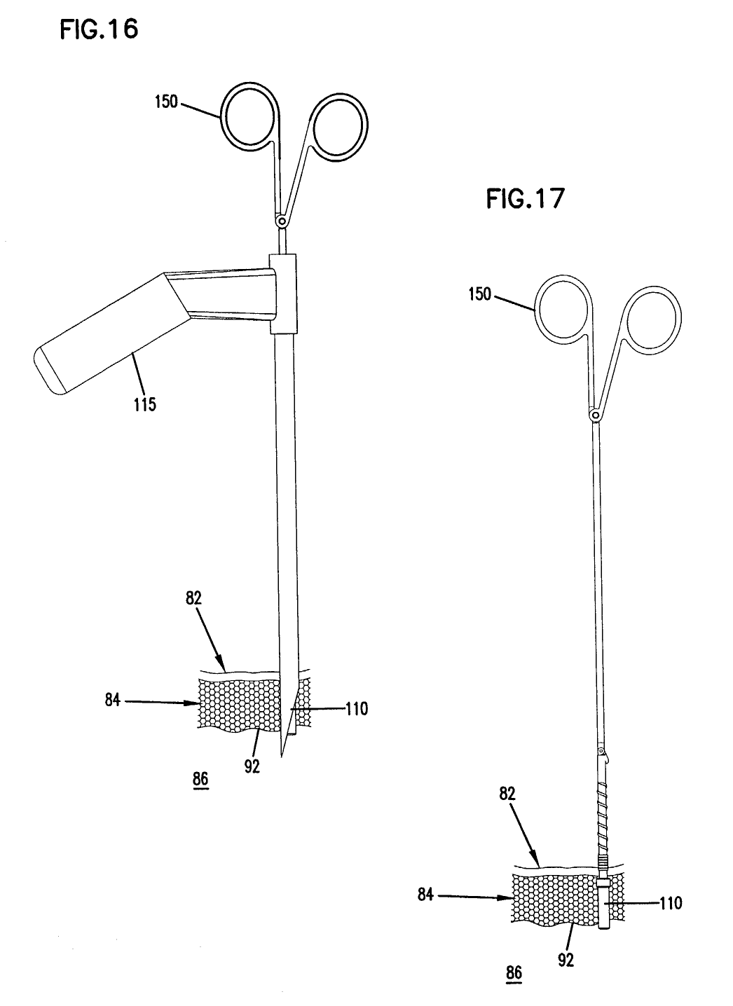

After sizing the myocardium thickness and selecting a delivery system having an implant of the appropriate size, the surgeon grasps tool 150 connected to second end 114 of implant 110 within delivery assembly 10 and urges delivery assembly 10 through myocardium 84. As shown in FIG. 15, the insertion location is offset from the occluded vessel. Delivery assembly 10 urges the tissue of myocardium 84 apart to form an opening through myocardium 84 sufficient to pass rigid portion 120 of implant 110. Implant delivery assembly 10 is positioned in myocardium 84 such that part-line 111 of sheath 100 faces distally from the obstruction. FIG. 16 shows sheath 100 inserted within myocardium 84. In some embodiments, a pre-formed guide opening can be provided through the myocardium (e.g., with a needle) to facilitate insertion of sheath 100. Also, in some embodiments, a guide wire can be pre-inserted through the myocardium and used to guide sheath 100.

To remove sheath 100 from around conduit 110, the surgeon grasps handle 115 of myocardial sheath 100 and pulls sheath 100 out of myocardium 84. Myocardial sheath 100 splits open at part-line 111 and myocardial sheath 100 flexes open to permit sheath 100 to clear implant 110 leaving rigid portion 120 and sleeve 122 (if present) of implant 110 within myocardium 84 (see FIG. 17).

After removal of sheath 100, flexible portion 130 may be communicated with vessel 82 via an end to side anastomosis. Flexible portion 130 may secured to vessel 82 by suturing or alternatively, by using an anastomosis device 140 such as that shown in FIGS. 4-7. During the procedure an incision is formed in a wall 83 of vessel 82. The surgeon grasps tool 150 holding the anastomosis device 140 at second 114 end of implant 110 in a compressed orientation. The surgeon inserts end 114 of the flexible portion 130 comprising anastomosis device 140 into the incision made in wall 83 of vessel 82. Alternatively, if tool 150 has a blade 152 on working end 151 as shown in FIGS. 10 and 11, the incision in wall 83 of vessel 82 can be performed almost simultaneously with insertion end 114 of flexible portion 130 of conduit 110 through wall 83.

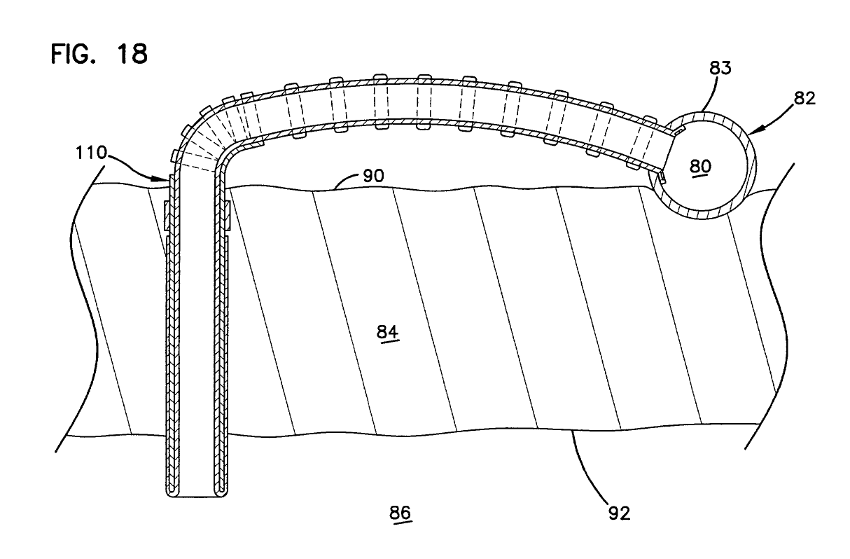

Once end 114 comprising anastomosis device 140 has been inserted through wall 83 within vessel 82, device 140 is released from tool 150 thereby allowing device 140 to self-expand within vessel 82, as shown in FIG. 18. Once expanded, blood pressure within vessel 82 preferably secures device 140 against wall 83 of vessel 82 thereby limiting the movement of device 140 and eliminating the need for sutures. If device 140 includes teeth 166, as shown in FIGS. 7-9, upon expansion, projection of teeth 166 beyond the bottom side of flange 145 embed within wall 83 of vessel 82 to create an auto-anastomosis. Teeth 166 include a degree of reverse curvature, as shown in FIGS. 8 and 9, which allow teeth 166 to penetrate wall 83 of vessel 82, but prevent teeth 166 from withdrawing once in place. It should be noted that in some situations it might be applicable to secure end 114 to vessel 82 using sutures or bio-glue.

Having disclosed the present invention in a preferred embodiment, it will be appreciated that modifications and equivalents may occur to one of ordinary skill in the art having the benefits of the teachings of the present invention. It is intended that such modifications shall be included within the scope of the claims are appended hereto.

What is claimed is:

1. A transmyocardial implant assembly, comprising:
a conduit for defining a blood flow path from a heart chamber to a coronary vessel, the conduit having a first end, a second end, a flexible portion and a rigid portion, the flexible portion including the second end which is sized to be received within a lumen of the coronary vessel, the rigid portion including the first end and sufficiently rigid to remain open during systole of a heart, the rigid portion sized to be inserted through and retained within a heart wall,
a sheath including an outer wall defining an inner diameter, at least a portion of the conduit releasably held within the inner diameter of the sheath; and
a tool extending within the sheath that is releasably attached to the second end of the conduit.

2. The transmyocardial implant assembly according to claim 1, wherein the assembly further comprises a handle connected to the sheath to facilitate insertion of the assembly into the heart wall.

3. The transmyocardial implant assembly according to claim 1, wherein the second end of the conduit comprises an auto-anastomosis device.

4. The transmyocardial implant assembly according to claim 3, wherein the autoanastomosis device is movable between an expanded orientation and a compressed orientation, has a resilient construction that biases the auto-anastomosis device toward the expanded orientation, and is adapted to be secured to the coronary vessel when in the expanded position.

5. The transmyocardial implant assembly of claim 4, wherein the auto-anastomosis device includes at least one tooth to mechanically engage a wall of the coronary vessel to secure the second end of the conduit to the coronary vessel when the autoanastomosis device is in an expanded orientation.

6. The transmyocardial implant assembly of claim 1, wherein the sheath is sufficiently rigid to be pushed though the heart wall.

7. The transmyocardial implant assembly according to claim 1, wherein the sheath includes a part line along a length of the sheath, the sheath separating at the part line to remove the sheath from the heart wall and to clear the conduit upon implantation.

8. The transmyocardial implant assembly according to claim 1, wherein a first end of the sheath is adapted to penetrate and bore through the heart wall.

9. The transmyocardial implant assembly according to claim 1, wherein the tool is a forceps.

10. The transmyocardial implant assembly according to claim 1, wherein the forceps includes a blade at a working end.

11. The transmyocardial implant assembly according to claim 1, wherein the conduit, sheath, and tool are a part of a kit contained within a sterile package.

12. The transmyocardial implant assembly according to claim 11, wherein the kit including the conduit, sheath, and tool further comprises a handle for holding the assembly and facilitating insertion of the assembly into the heart wall.

13. A method for forming a blood flow path from a heart chamber to a coronary vessel, comprising the steps of:
providing a transmyocardial implant delivery system with a sheath;
inserting sheath through a heart wall of the heart chamber at a location offset from the coronary vessel;
removing the sheath from the heart wall, leaving a hollow conduit in place within the heart wall, a first end of the conduit in fluid communication with the heart chamber; and
connecting a second end of the conduit to the coronary vessel.

14. The method of claim 13, wherein the conduit includes a rigid portion adjacent the first end within the heart wall which is sufficiently rigid to remain open during systole and diastole.

15. The method of claim 13, wherein the sheath comprises a part line along a length of the sheath, the sheath separating at the part line to remove the sheath from the heart wall and to clear the conduit after placing the transmyocardial implant delivery assembly in the heart wall.

16. The method according to claim 13, the method further comprising the steps of:
providing an incision in the coronary vessel;
inserting the second end of the conduit within a lumen of the coronary vessel; and
securing the second end within the lumen of the coronary vessel.

17. The method according to claim 16, wherein the second end of the conduit is releasably held by a working end of a tool within the sheath and is directed to coronary vessel by the tool upon removal of the sheath from the heart wall.

18. The method of claim 17, wherein a blade on the working end of the tool provides the incision.

19. The method according to claim 13, wherein the second end includes an anastomosis device having a compressible flange, wherein the anastomosis device is used to secure the second end of the conduit within a lumen of the coronary vessel.

20. The method according to claim 19 further comprising the steps of:
providing an incision in the coronary vessel;
compressing the flange to a compressed orientation;
inserting the second end including the compressed flange through the incision;
expanding the flange from the compressed orientation to an expanded orientation after the flange has been inserted through the incision; and
securing the expanded flange to the coronary vessel.

21. The method according to claim 10, wherein the second end of the conduit is releasably held by a working end of a tool within the sheath and is directed to coronary vessel by the tool upon removal of the sheath from the heart wall.

22. The method of claim 21, wherein a blade on the working end of the tool provides the incision.

23. The method according to claim 19 wherein the anastomosis device is an autoanastomosis device with the flange biased toward an expanded orientation and the method further comprising the steps of:
providing an incision in the coronary vessel;
compressing the flange to a compressed orientation;
inserting the second end including the compressed flange through the incision into the lumen of the coronary vessel;
releasing the flange after the flange has been inserted through the incision, the flange returning to the expanded orientation and securing the second end in fluid communication with the lumen of the coronary vessel.
